(19) United States
(12) Patent Application Publication
(10) Pub. No.: US 2008/0228010 A1
(43) Pub. Date: Sep. 18, 2008

(54) Alkoxylation process for preparing ether alkanol derivatives of phenolic compounds

(76) Inventors: Shenghong A. Dai, Taichung (TW); Hsing-Yo Lin, Taichung (TW); Chien-Sheng Wen, Taichung (TW)

Correspondence Address: TROXELL LAW OFFICE PLLC, 5205 LEESBURG PIKE, SUITE 1404, FALLS CHURCH, VA, 22041, US

(21) Appl. No.: 11/978,560

(22) Filed: Oct. 30, 2007

(30) Foreign Application Priority Data
Oct. 31, 2006 (TW) 095140299

(51) Int. Cl. C07C 41/09

(52) U.S. Cl. 568/648

(57) ABSTRACT

This invention provides an alkoxylation process for creating alkoxylating chain extension of phenolic compounds by reacting phenolic compounds with alkylene glycol and urea in the presence of suitable catalysts to obtain alkoxylated compounds, which may be used as polymer intermediates, especially for use as raw materials to synthesize polyurethane (PU) or polyester.

### FIELD OF THE INVENTION

This invention relates to an alkoxylation process for preparing ether alkanol derivatives of phenolic compounds, and more specifically to an alkoxylation process performed by reacting phenolic compounds with alkylene glycol and urea in the presence of suitable catalysts to obtain alkoxylated alcohols, which may be used as polymer intermediates.

### BACKGROUND OF THE INVENTION

Modifying properties of ethylene ether alcohols and propylene ether alcohols by performing chain extension to introduce hydrophilic group or hydrophobic group have played an important role in synthesis chemistry and the synthesis of polymer material. In industry, chain extension of hydroxyl group containing compounds (e.g., phenolic, amine or alcohol compounds) into poly(ether alkanol)s is performed by an alkoxylation reaction, i.e., reacting with ethylene oxide or propylene oxide in the presence of catalysts (e.g., sodium hydroxide or potassium hydroxide) at a temperature of about 140° C. to about 160° C. under a high pressure. This is a well-established technique widely applied for producing the materials used in the preparation of surfactants, detergents and polyols for polyurethane production.

However, it is disadvantageous to perform alkoxylation reaction by ethylene oxide or propylene oxide because of inconvenience in securing transportation of the relatively reactive (unstable) ethylene oxide and environmental pollution resulting from halogens required in the preparation of propylene oxide. In addition, there is a public safety issue associated with the high-pressure alkoxylation process using ethylene oxide/propylene oxide because of their relatively high vapor pressure at the reaction temperature of around 140° C. and above.

In a study published by Soos et al. (Journal of Polymer Science, Part A: Polymer Science (1999, 545-550)), bisphenol A (BPA) was found to react with ethylene carbonate or propylene carbonate in the presence of catalysts (e.g., K₂CO₃ or KHCO₃) at a temperature of about 170° C. to about 200° C. to produce ether diols of BPA with concurrent evolution of carbon dioxide. The ether diols of BPA have a structure similar to that of an ethylene oxide adduct or a propylene oxide adduct of bisphenol A except that the diols are shorter in chain length than the ether alcohols derived from EO/PO. The diols typically having one or less than three chain extension units are slightly different from the long-chain poly(ether alcohol)s produced by ring-opening reaction of ethylene oxide or propylene oxide having multiple repeating units. Therefore, they may have different applications.

Alkoxylation reaction using ethylene carbonate or propylene carbonate instead of more reactive ethylene oxide or propylene oxide can be performed relatively safely. However, it requires a higher production cost because ethylene carbonate and propylene carbonate are relatively expensive reagents.

Doya et. al. disclose (U.S. Pat. No. 5,349,077 (1994)) that ethylene carbonate or propylene carbonate can be synthesized by respectively reacting ethylene glycol or propylene glycol with urea in the presence of zinc oxide, with concurrent evolution of ammonia. This is a low-cost green method for synthesizing cyclic carbonates.

Accordingly, there is a need in the art for a safer, low-cost and more convenient one-pot atmospheric process of alkoxylation.

### SUMMARY OF THE INVENTION

Therefore, it is an object of the present invention to provide an new alkoxylation process for creating alkoxylating chain extension of phenolic compounds which is relatively simple, low-cost and environmentally friendly. This process avoids using gaseous reagent or halogen-derived compounds.

The present invention provides a novel process for chain extension of phenolic compounds by reacting with urea and alkylene glycol in the presence of suitable catalysts under atmospheric pressure. The ammonia and carbon dioxide released in the reaction can be recovered and then converted to urea as the material of alkoxylation reaction again. Since urea in essence acts as an intermediate that can be recycled in view of the reaction mechanism, the net reaction of the process can be regarded as dehydration condensation of a phenolic compound and an alkylene glycol to obtain an ether alcohol. Therefore, the process of the present invention is relatively simple, low-cost and environmentally friendly. The alkoxylation is also characterized by clean and selective derivatization of phenols with little side reactions.

An alkoxylation process in accordance with the invention is illustrated below:

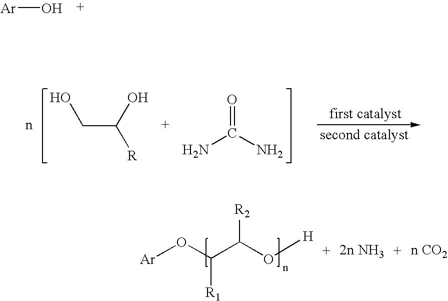

In this equation, 1≦n≦6; R represents H or C₁-C₁₆ alkyl; and R₁ represents H and R₂ represents H or C₁-C₁₆ alkyl, or R₂ represents H and R₁ represents H or C₁-C₁₆ alkyl groups.

The urea shown in this equation may be replaced by a carbonyl compound isoelectonically similar to urea such as the following formula (I), a carbonic acid ester having the following formula (II) or an urethane (carbamate) having the following formula (III):

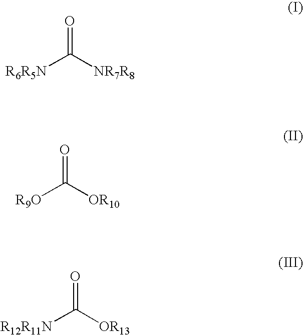

wherein R₅, R₆, R₇ and R₈ each independently represents H or C1-C6 alkyl, R₉ and R₁₀ each independently represents C1-C6 alkyl, R₁₁ and R₁₂ each independently represents H or C1-C6 alkyl, and R₁₃ represents C1-C6 alkyl. All these groups (R1˜13) could also be phenyl or substituted phenyl groups representing aromatic ureas for I, aromatic carbonates for II and aromatic carbamates for III respectively.

The ammonia and carbon dioxide shown in the equation can be re-converted to urea for use in the reaction again.

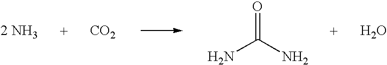

The alkoxylation reaction of the present invention may be performed at a temperature of about 100° C. to about 250° C. under normal atmospheric pressure. Any suitable phenolic compound can be use in the present invention. Preferably, the phenolic compounds are bisphenol A, hydroquinone, resorcinol or poly-phenols such as novalac. The alkylene glycol is preferably ethylene glycol, 1,2-propylene glycol, 1,2-butylene glycol, 1,2-pentylene glycol, 1,2-hexylene glycol or mixture thereof. The first catalyst is preferably NaOH, KOH, Na₂CO₃, LiCO₃, K₂CO₃, NaHCO₃, KHCO₃ or t-BuOK. The second catalyst is preferably ZnO, MgO or T-12.

The stoichiometry of the reaction requires about 2 to about 50 equivalents of alkylene glycol for each phenolic group, about 0.001 to about 0.5 equivalent of the first catalyst, about 0.001 to about 0.5 equivalent of the second catalyst, and/or about 1 to about 20 equivalents of urea per hydroxyl group of the phenolic compound.

Although the reaction is preferably carried out at atmospheric pressure, however, it could also be done under pressure if needed.

According to the present invention, the phenolic compound may premixed with alkylene glycol at a temperature of about 60° C. to about 160° C. to form a homogeneous mixture solution which then reacts with urea in the presence of the catalysts at a temperature of about 120° C. to about 230° C.

### DETAILED DESCRIPTION OF THE PREFERRED EMBODIMENT

Taking the alkoxylation of BPA with alkylene glycol as an example, the present invention may be embodied by the following scheme:

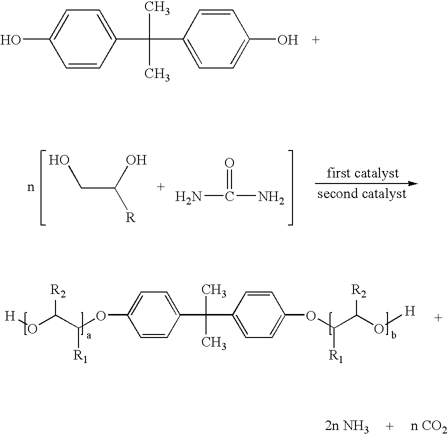

In this scheme, 2≦a+b≦6; n=a+b; R represents H or C1-C4 alkyl; and R₁ represents H and R₂ represents H or C1-C4 alkyl, or R₂ represents H and R₁ represents H or C1-C4 alkyl.

Taking the alkoxylation of hydroquinone with alkylene glycol as another example, the present invention may be embodied by the following scheme:

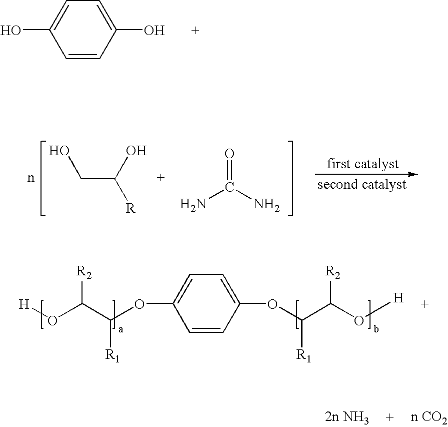

In this scheme, 2≦a+b≦6; n=a+b; R represents H or C1-C4 alkyl; and R₁ represents H and R₂ represents H or C1-C4 alkyl, or R₂ represents H and R₁ represents H or C1-C4 alkyl.

Several examples are embodied below to show the detailed reaction conditions and results, wherein examples 1-8 utilize BPA as the major reagent, and examples 9-10 utilize hydroquinone as the major reagent.

### EXAMPLE 1

To a three-neck 300 mL round bottom flask, equipped with a magnetic stirrer, thermometer, and reflux condenser, was charged with BPA (22.8 g, 0.1 mol), ethylene glycol (24.8 g, 0.4 mol) and Na₂CO₃ (0.2 g, 1.88 mmol). Then the reaction mixture was heated to 100° C. under a nitrogen atmosphere until BPA is dissolved. Thereafter, the reaction mixture was charged with urea (18.1 g; 0.3 mol) and ZnO (0.2 g, 2.46 mmol). The mixture was heated at 175° C. for 2 hours, and then cooled to room temperature. The excess ethylene glycol was removed by vacuum distillation. The distillation residue was extracted with ether, washed with water and dried by magnesium sulfate. Organic solvent was removed from the filtered organic phase by a vacuum evaporator to afford a crude product (29.2 g, 88% yield). ¹H NMR (R=H, a=1, b=1), δ 1.63 (s, 6H), 2.29 (s, 2H, —OH), 3.91 (t, 4H), 4.03 (t, 4H), 6.79-7.16 (dd, 8H, aromatics). The crude product was purified by re-crystallization with alcohol solution to afford desired product (27.8 g). Melting point 110° C.

### EXAMPLE 2

To a three-neck 500 mL round bottom flask, equipped with a magnetic stirrer, thermometer, and reflux condenser, was charged with BPA (22.8 g, 0.1 mol), propylene glycol (30.4 g, 0.4 mol) and Na₂CO₃ (0.2 g, 1.88 mmol). Then the reaction mixture was heated to 160° C. under a nitrogen atmosphere until BPA is dissolved. Thereafter, the reaction mixture was charged with urea (18.0 g; 0.3 mol) and ZnO (0.2 g, 2.46 mmol). The mixture was heated at 185° C. for 3 hours, and then cooled to room temperature. The excess propylene glycol was removed by vacuum distillation. The distillation residue was extracted with ethyl acetate and dried by magnesium sulfate. Organic solvent was removed from the filtered organic phase by a vacuum evaporator to afford desired product (33.8 g, 99% yield). ¹H NMR (R=CH₃. a=1. b=1). δ=1.25 (d. 6H), 1.63 (s. 6H), 1.98 (s. 2H. —OH), 3.71-4.25 (m. 6H), 6.78-7.16 (dd. 8H. aromatics).

### EXAMPLE 3

To a three-neck 500 mL round bottom flask, equipped with a magnetic stirrer, thermometer, and reflux condenser, was charged with BPA (22.8 g, 0.1 mol), ethylene glycol (50 g, 0.8 mol) and potassium carbonate (0.2 g, 1.45 mmol). Then the reaction mixture was heated to 160° C. under a nitrogen atmosphere until BPA is dissolved. Thereafter, the reaction mixture was charged with urea (36 g; 0.6 mol) and ZnO (0.4 g, 4.91 mmol). The mixture was heated at 190° C. for 3 hours, and then cooled to room temperature. The excess ethylene glycol was removed by vacuum distillation. The distillation residue was extracted with ethyl acetate and dried by magnesium sulfate. Organic solvent was removed from the filtered organic phase by a vacuum evaporator to afford desired product (33.1 g). (R=H. n=a+b=5.4) In this embodiment, the chain extension length (n) is estimated through ¹H NMR integration analysis. Specifically, the total alkoxylation protons (N) is obtained by comparison of the areas of aromatic protons (δ=6.8-7.2) to the areas of aliphatic protons (linked next to oxygen, —OCH₂CH₂O—, δ=3.4-4.2), and the chain extension length (n) is obtained by dividing N by 4 (since there are four aliphatic protons in each chain extension unit).

### EXAMPLE 4

To a three-neck 500 mL round bottom flask, equipped with a magnetic stirrer, thermometer, and reflux condenser, was charged with BPA (22.9 g, 0.1 mol), propylene glycol (76.3 g, 1.0 mol) and potassium carbonate (0.4 g, 2.9 mmol). Then the reaction mixture was heated to 110° C. under a nitrogen atmosphere until BPA is dissolved. Thereafter, the reaction mixture was charged with urea (48.0 g; 0.8 mol) and ZnO (0.4 g, 4.91 mmol). The mixture was heated at 185° C. for 6 hours, and then cooled to room temperature. The excess propylene glycol was removed by vacuum distillation. The distillation residue was extracted with ethyl acetate and dried by magnesium sulfate. Organic solvent was removed from the filtered organic phase by a vacuum evaporator to afford desired product (51.4 g). (R=CH₃. n=a+b=5.1). In this embodiment, the chain extension length (n) is also estimated through ¹H NMR integration analysis except that the chain extension length (n) is obtained by dividing the total alkoxylation protons (N) by 3 (since there are three aliphatic protons (linked next to oxygen, —OCH₂CHRO—, δ=3.4-4.2) in each chain extension unit).

Reagents, quantities, and reaction temperature of Examples 1-4 are listed in Table 1.

TABLE 1

| | Phenolic | | First | Mixing | | | Reaction |
|---|---|---|---|---|---|---|---|
| | Compound | Glycol | Catalyst | Temp. | Urea | Zinc Oxide | Temp. |
| Example | (mol) | (mol) | (mmol) | (° C.) | (mol) | (mmol) | (° C.) |
| 1 | BPA | Ethylene | Sodium | 100 | 0.3 | 2.46 | 175 |
| | (0.1) | Glycol | Carbonate | | | | |
| | | (0.4) | (1.88) | | | | |
| 2 | BPA | Propylene | Sodium | 160 | 0.3 | 2.46 | 185 |
| | (0.1) | Glycol | Carbonate | | | | |
| | | (0.4) | (1.88) | | | | |
| 3 | BPA | Ethylene | Potassium | 160 | 0.6 | 4.91 | 190 |
| | (0.1) | Glycol | Carbonate | | | | |
| | | (0.8) | (1.45) | | | | |
| 4 | BPA | Propylene | Potassium | 110 | 0.8 | 4.91 | 185 |
| | (0.1) | Glycol | Carbonate | | | | |
| | | (1.0) | (2.9) | | | | |

### EXAMPLE 5-8

Repeat the reaction steps of Example 2 wherein reagents, quantities, reaction temperature and time are shown in Table 2.

TABLE 2

| | | 1,2- | | | | Reaction | Reaction |
|---|---|---|---|---|---|---|---|
| | | Propylene | | Sodium | Zinc | Temp. | Time |
| Example | BPA | Glycol | urea | Carbonate | Oxide | (° C.) | (hr) |
| 5 | 0.2 mol | 0.8 mol | 0.6 mol | 3.85 mmol | 5 mmol | 120~205 | 8 |
| 6 | 0.1 mol | 0.4 mol | 0.3 mol | 3.85 mmol | 2.5 mmol | 120~185 | 4.5 |
| 7 | 0.1 mol | 0.6 mol | 0.3 mol | 1.93 mmol | 2.5 mmol | 100~180 | 4 |
| 8 | 0.1 mol | 0.4 mol | 0.3 mol | 1.93 mmol | 2.5 mmol | 160~185 | 3 |

### EXAMPLE 9

To a three-neck 250 mL round bottom flask, equipped with a magnetic stirrer, thermometer, and reflux condenser, was charged with hydroquinone (11.0 g, 0.1 mol), ethylene glycol (21.1 g, 0.34 mol), sodium carbonate (0.2 g, 1.88 mmol), urea (15.0 g, 0.25 mol) and ZnO (0.2 g, 2.46 mmol). Then the reaction mixture was heated to 180° C. for 3 hours, and then cooled to room temperature. The reaction mixture was extracted with ethyl acetate. Organic solvent was removed from the filtered organic phase by a vacuum evaporator to afford crude product (19.4 g, 98% yield). ¹H NMR (R=H. a=1. b=1), d=2.04 (s. 2H. —OH), 3.79 (t. 4H), 3.95 (t. 4H), 6.84 (s. 4H. aromatics).

### EXAMPLE 10

To a three-neck 500 mL round bottom flask, equipped with a magnetic stirrer, thermometer, and reflux condenser, was charged with hydroquinone (27.5 g, 0.25 mol), ethylene glycol (76.3 g, 1.0 mol) and sodium carbonate (0.4 g, 3.77 mmol). Then the reaction mixture was heated to 110° C. under a nitrogen atmosphere until hydroquinone is dissolved. Thereafter, the reaction mixture was charged with urea (45 g; 0.75 mol) and ZnO (0.4 g, 4.91 mmol). The mixture was heated at 180° C. for 6 hours, and then cooled to room temperature. The distillation residue was extracted with ethyl acetate. Organic solvent was removed from the filtered organic phase by a vacuum evaporator to afford crude product (61.5 g, 100% yield). ¹H NMR (R=CH₃. a=1. b=1). δ=1.19-1.28 (m. 6H), 2.55 (s. 2H. —OH), 3.69-4.41 (m. 6H), 6.84 (s. 4H. aromatics).

Chain extension results of the present invention are investigated by NMR. The δ value of the first chain extension unit is 4.0 ppm adjacent to benzene ring in ¹H NMR. The δ value of the second chain extension unit is 3.6 ppm in ¹H NMR. The δ value of the other chain extension units are shifted to up-field in ¹H NMR.

The yield and the chain extension length of examples according to the present invention are listed in Table 3.

TABLE 3

| | | Chain Extension Length |
|---|---|---|
| Example | Yield (%) | (a + b) |
| 1 | 88 | 2 |
| 2 | 99 | 2 |
| 3 | 71^{a} | 5.4^{c} |
| 4 | 98^{a} | 5.1^{c} |
| 5 | 99 | 2 |
| 6 | 99 | 2 |
| 7 | 99 | 2 |
| 8 | 99 | 2 |
| 9 | 98 | 2 |
| 10 | >100^{b} | 2 |

^{a}determined as a percentage of the product quantity obtained to the theoretically obtainable quantity estimated based on the average chain extension theory.
^{b}yiled over 100% because there is a portion of carbonic acid ester existed in the product due to incompletely release of carbon dioxide.
^{c}estimated through NMR integration analysis.

From the results listed above, we have shown that one-pot reaction can be used to accomplish chain extension reaction of phenolic compounds by reacting phenolic compounds having hydroxyl group (e.g., BPA and phenol) with alkylene glycols (e.g., ethylene glycol and propylene glycol) and urea in the presence of a first catalyst (e.g., Na₂CO₃ and K₂CO₃) and a second catalyst (e.g., ZnO and MgO). A series of ether diols may be obtained by reacting with corresponding alkylene glycols.

What is claimed is:

1. A process for producing a compound having the following formula I by reacting a phenolic compound with alkylene glycol and chemical A in the presence of first and second catalysts at a temperature of about 100° C. to about 250° C., 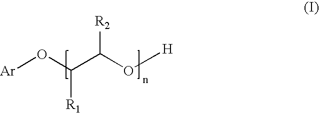
wherein:
Ar represents a substituted or unsubstituted aryl group or heterocyclic aryl group,
1≦n≦6;
R₁ represents H and R₂ represents H or C₁-C₁₆ alkyl, or R₂ represents H and R₁ represents H or C₁-C₁₆ alkyl;
chemical A represents urea or carbonyl compounds iso-electronic with urea having the following formula (I), a carbonic acid ester having the following formula (II) or an amine ester having the following formula (III): 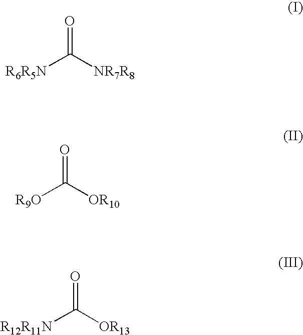
wherein R₅, R₆, R₇ and R₈ each independently represents H or C₁-C₆ alkyl, R₉ and R₁₀ each independently represents C₁-C₆ alkyl, R₁ and R₁₂ each independently represents H or C1-C6 alkyl, and R₁₃ represents C1-C6 alkyl.

2. The process according to claim 1, wherein the phenolic compound is selected from the group consisting of bisphenol A, hydroquinone and resorcinol.

3. The process according to claim 1, wherein the alkylene glycol is selected from the group consisting of ethylene glycol, 1,2-propylene glycol, 1,2-butylene glycol, 1,2-pentylene glycol, 1,2-hexylene glycol and mixture thereof.

4. The process according to claim 1, wherein the first catalyst is selected from the group consisting of NaOH, KOH, Na₂CO₃, LiCO₃, K₂CO₃, NaHCO₃, KHCO₃ and t-BuOK.

5. The process according to claim 1, wherein the second catalyst is selected from the group consisting of ZnO, MgO and T-12.

6. The process according to claim 1, wherein the stoichiometry of the reaction requires about 2 to about 50 equivalents of alkylene glycol per hydroxyl group of the phenolic compound.

7. The process according to claim 1, wherein the stoichiometry of the reaction requires about 0.001 to about 0.5 equivalent of the first catalyst per hydroxyl group of the phenolic compound.

8. The process according to claim 1, wherein the stoichiometry of the reaction requires about 0.001 to about 0.5 equivalent of the second catalyst per hydroxyl group of the phenolic compound.

9. The process according to claim 1, wherein the stoichiometry of the reaction requires about 1 to about 20 equivalents of urea per hydroxyl group of the phenolic compound.

10. The process according to claim 1, wherein the reaction pressure ranges from 1 to 100 atm.

11. The process according to claim 1, wherein the phenolic compound reacts with alkylene glycol in the presence of the first catalyst at a temperature of about 60° C. to about 160° C. to form a mixture solution which then reacts with urea in the presence of the second catalyst at a temperature of about 155° C. to about 230° C.
